(19) United States
(12) Patent Application Publication
(10) Pub. No.: US 2008/0175902 A1
(43) Pub. Date: Jul. 24, 2008

(54) Methods for slowing the progression of multiple sclerosis

(75) Inventor: John Zajicek, Plymouth (GB)

Correspondence Address: Davidson, Davidson & Kappel, LLC, 485 7th Avenue, 14th Floor, New York, NY, 10018, US

(73) Assignee: University of Plymouth, Plymouth (GB)

(21) Appl. No.: 11/998,373

(22) Filed: Nov. 28, 2007

Related U.S. Application Data
(60) Provisional application No. 60/861,850, filed on Nov. 30, 2006.

(51) Int. Cl. A61K 9/48; A61K 31/352; A61P 25/00

(52) U.S. Cl. 424/456; 514/454

(57) ABSTRACT

Methods are provided herein for slowing the progression of multiple sclerosis comprising administering a therapeutically effective amount of a cannabinoid to a patient suffering from MS.

This application claims the benefit of U.S. Provisional Patent Application No. 60/861,850, filed on Nov. 30, 2006, the disclosure of which is hereby incorporated by reference in its entirety.

### FIELD OF THE INVENTION

The present invention relates to methods of preventing the progression of multiple sclerosis (MS).

### BACKGROUND OF THE INVENTION

Multiple sclerosis or MS is a disease that affects the brain and spinal cord resulting in loss of muscle control, vision, balance, sensation (such as numbness) or thinking ability.

With MS, the nerves of the brain and spinal cord are damaged by one's own immune system. In MS, the immune system attacks the brain and spinal cord, the two components of the central nervous system. The central nervous system is made up of nerves that act as the body's messenger system. Each nerve is covered by a fatty substance called myelin, which insulates the nerves and helps in the transmission of nerve impulses, or messages, between the brain and other parts of the body. MS gets its name from the buildup of scar tissue (sclerosis) in the brain and/or spinal cord. The scar tissue or plaques form when the protective and insulating myelin covering the nerves is destroyed, a process called demyelination. Without the myelin, electrical signals transmitted throughout the brain and spinal cord are disrupted or halted. The brain then becomes unable to send and to receive messages. It is this breakdown of communication that causes the symptoms of MS. Although the nerves can regain myelin, this process is not fast enough to outpace the deterioration that occurs in MS.

There are a variety of medications available that can reduce the frequency and severity of MS symptoms in some people with MS. Symptoms may be divided into three categories: primary, secondary, and tertiary. Primary symptoms are a direct result of the demyelination process. This impairs the transmission of electrical signals to muscles (to allow them to move appropriately) and the organs of the body (allowing them to perform normal functions.) The symptoms include: weakness, tremors, tingling, numbness, loss of balance, vision impairment, paralysis, and bladder or bowel problems.

Secondary symptoms result from primary symptoms. For example, paralysis (a primary symptom) can lead to bedsores (pressure sores) and bladder or urinary incontinence problems can cause frequent, recurring urinary tract infections. These symptoms can be treated, but the ideal goal is to avoid them by treating the primary symptoms.

Tertiary symptoms are the social, psychological, and vocational complications associated with the primary and secondary symptoms. Depression, for example, is a common problem among people with MS.

The course of multiple sclerosis varies for each person. Because of this uncertainty, doctors often tell their patients that they “probably” or “possibly” have MS. Diagnosis is based on the combination of problems, patterns of recurrence, which systems are impaired and your test results. There is no way to predict how each person's condition will progress. It often takes years before a doctor can be certain of an MS diagnosis and have some idea on how the disease will progress. There are three main courses that MS takes:

Relapsing-remitting MS: characterized by unpredictable acute attacks, called “exacerbations,” with worsening of symptoms followed by full, partial or no recovery of some function. These attacks appear to evolve over several days to weeks. Recovery from an attack takes weeks sometimes months. The disease does not worsen in the periods between the attacks. This pattern usually occurs early in the course of MS in most people.

Primary-progressive MS: characterized by a gradual but steady progression of disability, without any obvious relapses and remissions. This form of disease occurs in just 15% of all people with MS, but it is more common in people who develop the disease after the age of 40.

Secondary-progressive MS: initially begins with a relapsing-remitting course, but later evolves into progressive disease. The progressive part of the disease may begin shortly after the onset of MS, or it may occur years or decades later.

A true exacerbation of MS is caused by an area of inflammation (swelling) in the nerves of the brain and spinal cord system followed by something called demyelination, which is the destruction of myelin. The myelin is the fatty sheath that surrounds and protects the nerve fibers. An exacerbation of MS may be mild and not cause a noticeable impairment in functioning or may significantly interfere with a person's daily life. Exacerbations usually last from several days to several weeks, although they may extend into months.

Current therapy intended to slow MS progression has many difficulties. A number of drugs have been shown to slow the progression of MS in some people. These drugs work by suppressing, or altering, the activity of the body's immune system. Thus, these therapies are based on the theory that MS is, at least in part, a result of an abnormal response of the body's immune system that causes it to attack the myelin surrounding nerves. These medications do not cure MS, but they do reduce the frequency and severity of attacks and the development of new brain lesions. In addition, they slow down the progression of MS, reducing future disability. Examples include Avonex® (interferon beta-1a), Betaseron® (interferon beta-1b), Copaxone® (glatiramer acetate), Novantrone® (mitoxantrone), Rebif® (interferon beta-1a), Tysabri® (natalizumab). The interferon drugs can lead to adverse effects such as liver toxicity, hematological abnormalities, improper thyroid function, sadness, anxiety, irritability, guilt, poor concentration, confusion and difficulties sleeping or eating. Copaxone can lead to chest pain or tightness, heart palpitations, anxiety, flushing and difficulty breathing. Novantrone can lead to fever, chills, sore throat or cough, sores on lips or in mouth, shortness of breath, stomach pain, vomiting, diarrhea, painful or difficult urination, uneven or rapid heartbeat, swollen feet or ankles, unusual bleeding or bruising, and pain, swelling, redness, or irritation at the injection site, menstrual irregularities and hair loss. Tysabri can lead to infections, allergic reactions, depression, gall bladder disorders, headache, fatigue, joint pain and menstrual problems.

Other undesirable characteristics of these drug therapies include unpleasantness, pain and discomfort associated with injections, high cost and drug stability.

Cannabinoid medical use has been discussed in the art. For example, see: Petro, D. J., et al; “Treatment of Human Spasticity with Δ9-Tetrahydrocannabinol”; J. Clin. Pharmacol, 1981, 21: 413S-416S; Kumar R. N., et al; “Pharmacological actions and therapeutic uses of cannabis and cannabinoids”; Anesthesia, 2001, 56: 1059-1068; Pertwee, R. G., “Cannabinoids and Multiple Sclerosis”; Pharmacology and Therapeutics, 2002, 95: 165-174; Baker, D., et al; “Cannabinoids control spasticity and tremor in a multiple sclerosis model”; Nautre, 2000, 6773: 84-86. In Killestein, J., et al; “Safety Tolerability, and efficacy of orally administered cannabinoids in MS”; Neurology, 2002, 58: 1404-1407; the benefit of cannabis plant extract and THC in patients with MS over a 4 week period was studied and found that there was no significant changes in Ashworth scores. In Zajicek, J, et al; “Cannabinoids for treatment of spasticity and other symptoms related to multiple sclerosis (CAMS study): multicentre randomized placebo-controlled trial”; Lancet, 2003, 362: 1517-1526; the benefit of cannabis plant extract and Δ9-THC administered to patients in a 15 week trial was tested. This study also did not yield results indicating that cannabinoids including dronabinol slowed progression of MS.

In light of the above experiments, there is still a need in the art for a safe and effective method for slowing the progression of MS.

Delta-9-Tetrahydrocannabinol (also known as THC, dronabinol, Δ9-THC and D9-THC) is a naturally occurring compound and is the primary active ingredient in the controlled substance marijuana. Marijuana refers to the dried flowers and leaves of Cannabis Sativa, the hemp plant. These parts of the plant contain several compounds called cannabinoids (including dronabinol), that may help patients with certain disease conditions. Dronabinol has been approved by the Food and Drug Administration (FDA) for the control of nausea and vomiting associated with chemotherapy and, more recently, for appetite stimulation of AIDS patients suffering from wasting syndrome. Synthetic dronabinol has been utilized as a pharmaceutically active ingredient, and cannabis-based medicines using botanical sources of cannabis rather than synthetic THC are also known in the art. However, dronabinol is not currently approved for use in slowing the progression of MS.

### SUMMARY OF THE INVENTION

It is an object of the present invention to provide a method of slowing the progression of multiple sclerosis in a patient in need thereof.

The present invention is directed to the administration of a cannabinoid to human patients suffering from MS in order to slow the progression of the disease.

Accordingly, in one embodiment, the present invention provides a method of slowing the progression of MS. The method comprises administering to a subject suffering from MS a therapeutically effective amount of a cannabinoid, for example delta-9-THC.

According to methods of the present invention, a cannabinoid may be administered alone or in combination with one or more pharmaceutically effective carrier(s) or other pharmaceutically acceptable excipient(s) or additive(s).

In addition, a cannabinoid, for example dronabinol, can be administered concurrently or in succession with other medications which may treat the underlying disease state (MS) or which provide at least some alleviation of the symptoms of the disease, i.e. symptomatic therapies.

The method of the invention comprises administering a pharmaceutical composition comprising an effective amount of a therapeutically effective cannabinoid on a regular basis to an MS patient or a population of MS patients; the administration occurring over a period of time selected from the group consisting of at least about 16 weeks, at least about 27 weeks, at least about 40 weeks and at least about 52 weeks.

In certain preferred embodiments, the therapeutically effective cannabinoid used in the methods of the invention are selected from, e.g., one or more (i) natural cannabinoids that have been purified or modified; (ii) synthetically derived cannabinoids; (iii) semi-synthetic cannabinoids; (iv) esterified cannabinoids; (v) active metabolites of any of the foregoing, and (vi) pro-drugs of any of the foregoing. The therapeutically effective cannabinoid can also comprises (vii) mixtures of any of the foregoing. In certain preferred embodiments, the cannabinoid comprises D9-THC. In other preferred embodiments, the cannabinoid comprises THC-hemisuccinate.

In certain embodiments, the patient(s) display(s) an improvement in at least one indicator of progression of multiple sclerosis selected from the group consisting of Ashworth score, Rivermead Mobility Index value and timed 10 metre walk compared to a group of patients administered placebo over at least about 16 weeks.

By “at least about 16 weeks”, it is meant that a comparison can be made to find an improvement in a test score between the patients treated in accordance with the invention for, e.g., about 52 weeks, as compared to a population of patients receiving placebo, for example, over about 16 weeks; or over about 27 weeks; or over about 40 weeks; or over about 52 weeks, as the case may be.

By “administration . . . on a regular basis”, it is meant that the cannabinoid is administered to the MS patient(s) at regular dosing intervals such that therapeutic efficacy is maintained at steady-state. It will be appreciated by those skilled in the art that the dosing interval will be dependent on many variables, including but not limited to the choice of therapeutically effective cannabinoid(s), the pharmaceutical excipients incorporated with the cannabinoid(s) into the dosage form which is administered, the method of manufacture, the route of administration, etc.

In certain other embodiments, the patient displays an improvement in Ashworth score, compared to a group of patients administered placebo over at least about 16 weeks.

In still other embodiments of the invention, the patient displays an improvement in Rivermead Mobility index, compared to a group of patients administered placebo over at least about 16 weeks.

In further embodiments of the invention, the patient displays significant improvement in Rivermead Mobility index, compared to a group of patients administered placebo over at least about 16 weeks.

In still further embodiments, the patient displays an improvement in 10 metre walk time, compared to a group of patients administered placebo over at least about 16 weeks.

In certain embodiments, the patient displays a significant improvement in at least one symptom selected from the group consisting of pain, spasms, spasticity and sleep, compared to a group of patients administered placebo over at least about 16 weeks.

In accordance with the above objects, the invention is also directed to a method of slowing progression of multiple sclerosis wherein the composition is orally administered. In other embodiments, the composition is encapsulated in a gelatin or non-gelatin shell.

In other embodiments, the composition is a liquid solution or liquid suspension. In still other embodiments, the liquid is a hydrophobic material, a hydrophilic material or an amphiphilic material. In certain embodiments, the liquid is selected from the group consisting of an oil, water or an alcohol. In still other embodiments, the liquid is sesame oil.

The present invention is also directed to a method of slowing the progression of multiple sclerosis in a patient in need thereof comprising administering on a regular basis, an oral pharmaceutical composition comprising an effective amount of D9-THC, an oil and a capsule.

In accordance with the above methods, the present invention is also directed to a method wherein the patient exhibits an increase of about 0.2 to about 1.1 in Rivermead Mobility Index values at about 12-27 weeks of administration compared to a group of patients administered placebo over at least about 16 weeks.

In other embodiments, the patient exhibits an increase of about 0.25 to about 1.3 in Rivermead Mobility Index values at about 25-40 weeks of administration compared to a group of patients administered placebo over at least about 16 weeks.

In further embodiments, the patient exhibits an increase of about 0 to about 1.2 in Rivermead Mobility Index values at about 35-50 weeks of administration compared to a group of patients administered placebo over at least about 16 weeks.

In still further embodiments, the patient exhibits a change in Ashworth scores of from about −1 to about 2.5 at about 12-27 weeks of administration compared to a group of patients administered placebo over at least about 16 weeks.

In yet further embodiments, the patient exhibits a change in Ashworth scores of from about −5 to about 3.5 at about 25-40 weeks of administration compared to a group of patients administered placebo over at least about 16 weeks.

In certain other embodiments, the patient exhibits an increase in Ashworth scores of from about 0.2 to about 4 at about 35-50 weeks of administration compared to a group of patients administered placebo over at least about 16 weeks.

The present invention is also directed to methods in accordance with the above methods wherein the composition is administered in a dosage form selected from the group consisting of an intranasal solution, an intranasal suspension, an inhalant solution, an inhalant suspension, a parenteral solution, a parental suspension, a transdermal patch, a transdermal gel, a transdermal cream, a transdermal ointment, and a transdermal lotion.

In certain embodiments, the composition is administered in a dosage form selected from the group consisting of a tablet, a capsule, an oral inhalant, a nasal inhalant, an injectable, a transdermal, a sublingual, and a suppository. In still other embodiments, the administered dosage form is a soft gelatin capsule or HPMC capsule.

In certain other embodiments, the composition is administered in combination with one or more multiple sclerosis therapies.

In still other embodiments of the present invention, the composition is administered in an amount sufficient to provide from about 2.5 mg D9-THC to about 20 mg D9-THC per day. In other embodiments, the composition is administered in an amount sufficient to provide about 2.5 mg D9-THC per day. In certain embodiments, the composition is administered about 1 to about 4 times per day.

### BRIEF DESCRIPTION OF THE DRAWINGS

FIG. 1 shows changes in Ashworth scores from baseline to 13 weeks follow-up after initiation of the study, adjusted for ambulatory status and center effects.

FIG. 2 shows effect of ambulation on Ashworth scores by treatment group.

FIG. 3 shows changes in Ashworth scores by visit and treatment group.

FIG. 4 shows median 10 meter walk times by visit and treatment group.

FIG. 5 shows changes in Ashworth scores by visit and treatment group over the 52 week trial period.

FIG. 6 shows changes in the Rivermead Mobility Index values by visit and treatment group over the 52 week trial period.

### DETAILED DESCRIPTION

Although certain sections of this specification provide specific focus on dronabinol, the present invention is applicable to the class of pharmaceutically acceptable cannabinoids. For purposes of the present invention, the term “cannabinoid” includes naturally occurring and non-natural derivatives of cannabinoids which can be obtained by derivatization of natural cannabinoids and which are unstable like natural cannabinoids. In other words, the cannabinoid used in the formulations of the invention may be natural, semi-synthetic, or synthetic. The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms. The term “cannabinoid” is also meant to encompass derivatives that are produced from another compound of similar structure by the replacement of, e.g., substitution of one atom, molecule or group by another such as 11-hydroxy-delta-8-tetrahydrocannabinol and 11-hydroxy-delta-9-tetrahydrocannabinol. The term “cannabinoid”, as used in the present invention, further includes delta-8-tetrahydrocannabinol, delta-9-tetrahydrocannabinol, cannabidiol, olivetol, cannabinol, cannabigerol, nabilone, delta-9-tetrahydro cannabinotic acid, the non-psychotropic cannabinoid 3-dimethylnepty 11 carboxylic acid homologine 8. (J. Med. Chem. 35, 3135, 1992). The term cannabinoid also includes prodrugs of cannabinoids, as well as pharmaceutically acceptable salts and complexes of cannabinoids. An example of a suitable prodrug is THC-hemisuccinate.

The term “cannabinoid” is further meant to encompass natural cannabinoids that have been purified or modified, and synthetically derived cannabinoids, for example, United States Patent Application Publication 2005/0266108, hereby incorporated by reference in its entirety, describes a method of purifying cannabinoids obtained from plant material. The term cannabinoid is also meant to include the compounds described in U.S. Pat. No. 6,713,048, including levonantradol, (−)-HU-210, Win 55212-2, Anandamide, Methandamide, CP 55940, 0-1057, SR141716A, etc.). The disclosure of this patent is hereby incorporated by reference in its entirety.

In certain preferred embodiments of the present invention, the active ingredient (cannabinoid) comprises or consists essentially of Delta-9-tetrahydrocannabinol, also known as (and referred to herein as) dronabinol. Dronabinol is naturally-occurring and has been extracted from Cannabis saliva L. (marijuana). It has also been produced chemically as described in U.S. Pat. No. 3,668,224. Dronabinol is a light-yellow resinous oil that is sticky at room temperature, but hardens upon refrigeration. It turns to a flowable liquid when heated at higher temperatures. Dronabinol is insoluble in water. It has a pKa of 10.6 and an octanol-water partition coefficient: 6,000:1 at pH 7. Dronabinol is available in natural (extracted from plant) and synthetic forms. On the other hand, synthetic dronabinol may be utilized and may be synthesized using the starting materials, Olivetol and p-2,8-menthadien-2-ol (PMD).

The term “dronabinol” is further meant to encompass naturally occurring dronabinol, synthetically derived dronabinol, and synthetically modified dronabinol starting with a molecule obtained from a natural source for example, United States Patent Application Publication 2005/0171361, hereby incorporated by reference in its entirety, describes a method of extracting delta-9-THC acid from the plant material by chromatography and then synthetically converting it to dronabinol.

In certain preferred embodiments of the invention, the cannabinoid used in the formulation is esterified. Esterified forms of THC are described in U.S. Pat. No. 4,933,368 and in U.S. Pat. No. 5,389,375. Other useful polar esters are the hemi-ester of malonic acid and the alaninate ester of alanine. It has been reported, e.g., in U.S. Pat. Nos. 5,508,051 and 5,389,375, that salts of the terminal carboxylic acid group of the ester, for example, the N-methyl glutamine salt as well as the sodium and potassium salts are also useful. The descriptions of U.S. Pat. Nos. 4,933,368; 5,508,037; and 5,389,375, are incorporated herein by reference. These ester compounds are hydrolyzed in the blood stream releasing THC to provide a high degree of bioavailability of THC without regard to patient conditions and anomalies.

Oral THC is known to possess erratic absorption from the gastrointestinal tract, is subject to the first-pass effect resulting in heavy metabolism with production of high levels of 11-OH-delta-9-THC. It is reported that this 11-hydroxy metabolite is more potent agonist than delta-9-THC. The pro-drug THC hemisuccinate (THC-HS) has been formulated in a suppository base as described in U.S. Pat. Nos. 5,508,037 and 5,389,375, both of which are hereby incorporated by reference) in order to avoid this problem. Preliminary clinical investigations show promise for this formulation (Mattes, R. D.; Shaw, L. M.; Edling-Owens, J., Engleman, K.; and ElSohly, M. A.; Bypassing the first-pass effect for the therapeutic use of cannabinoids; Pharm., Biochem., Behav., 44(3):745-747, 1991; Mattes, R. D.; Engelman, K.; Shaw, L. M.; and ElSohly, M. A.; Bypassing the first-pass effect for the therapeutic use of cannabinoids, Pharmacol., Biochem., Behav., 49(1):187-195, 1994; Brenneisen, R.; Egli, A.; ElSohly, M. A.; Henn, V.; and Speiss, Y.; The effect of orally and rectally administered delta-9-tetrahydrocannabinol on spasticity: A pilot study with 2 patients; Inter. J. Clin. Pharmacol. and Therapeutics, 34(10):446-452, 1996; all of which are hereby incorporated by reference).

THC obtained by any means can be esterified by the reaction of THC with an organic acid, an organic acid halide or preferably organic acid anhydride in the presence of 4-amino-substituted pyridine alone or in admixture with an organic amine, or in any other manner known to those skilled in the art. U.S. Pat. No. 6,008,383 (Elsohly, et al.), hereby incorporated by reference, describes a process for converting dronabinol to a variety of ester analogs, which process is said to be economical and efficient. Therein, dronabinol is esterified by reaction with a carboxylic acid, an acid halide or an acid anhydride in the presence of a 4-aminopyridine either alone or in admixture with an organic amine such as a mono-, di-, or tri-alkyl amine.

In certain preferred embodiments, the cannabinoid comprises dronabinol hemisuccinate ester (THC-HS).

When compositions are used in a “therapeutically-effective amount” according to the present invention, this means that the dose of the therapeutic agent (or agents) provides a desired therapeutic effect over the course of the dosing interval. Such delivery is dependent on a number of variables including the time period for which the individual dosage unit is to be used, or the flux rate of the therapeutic agent into the systemic circulation of the subject. It will be understood, however, that specific dose levels of the therapeutic agents of the present invention for any particular subject may depend upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. It should be appreciated that the dosage to be administered will depend on several factors, including the particular agent that is administered, the route administered, the physical state of the particular agent, the condition of the particular subject, etc.

The dose administered to a subject, particularly a human subject, in the context of the present invention should be sufficient to affect a therapeutic response over a reasonable time frame. The dose will be determined by the strength of the particular compositions employed and the condition of the person, as well as the body weight of the person to be treated. The size of the dose also will be determined by the existence, nature, and extent of any adverse side-effects that might accompany the administration of a particular composition. A suitable dosage for internal administration is 0.01 to 100 mg/kg per day. A preferred dosage is 0.01 to 35 mg/kg per day. A more preferred dosage is 0.05 to 5 mg/kg per day. A suitable concentration of dronabinol in pharmaceutical compositions for oral administration is 0.05 to 15% (by weight). A preferred concentration is from 0.02 to 5%. A more preferred concentration is from 0.1 to 4%. More preferably, 0.03 to 0.06 mg/kg body weight per day is administered orally, and most preferably, a 2.5 mg oral dosage form is administered two times per day. The most preferred dosage for extracorporeal administration is in the range from about 0.1 mg/kg to 5 mg/kg of body weight per day. For the rectal, topical (including buccal and sublingual) or transdermal route of administration, the preferred dosage thereof (estimated as the base) is in the range 0.05 mg/kg to 20 mg/kg of body weight per day. Although dronabinol may be administered as needed, preferably, dronabinol is administered one to five times per day.

Where dronabinol is the active drug in a composition used according to methods of the instant invention, it is preferable that dronabinol will be present in such a composition in a total amount of about 0.5 mg to about 20 mg, preferably about 1 mg to about 15 mg, and more preferably about 2 mg to about 12 mg. Illustratively, such a composition will comprise about 2 mg, about 2.5 mg, about 5 mg or about 10 mg dronabinol.

Cannabinoids can be formulated into any pharmaceutically acceptable formulation for use in methods according to the present invention. The formulations used in the methods of the invention typically include a therapeutically effective amount of a cannabinoid together with one or more pharmaceutically acceptable excipients. Such excipients may include (but are not limited to), diluents, fillers, binders, lubricants, glidants, pH modifiers, stabilizers, disintegrants, carriers, vehicles, release modifying agents (e.g., extended release, enteric release, rapid release), viscositiy modifiers, solubilizers, colors, flavoring agents, taste-masking agents and sweeteners. Preferably, the excipients do not adversely affect the stability of the formulation. The formulations can be designed to deliver the medicament by any acceptable route of administration including oral, buccal, sublingual, subcutaneous, transdermal, intramuscular or intravenous, rectal, topical or inhalation administration.

Examples of pharmaceutically acceptable binders are natural and synthetic gums, saccharides and polysaccharides such as acacia, alginic acid and salts thereof, cellulosic materials such as alkyl celluloses, alkoxy celluloses and salts thereof such as methylcellulose, ethylcellulose, hydroxypropyl methylcellulose, hydroxyethyl cellulose, hydroxypropyl cellulose, microcrystalline cellulose, polymers and copolymers, magnesium aluminum silicate, polyethylene glycol, polysaccharide acids, bentonites, gelatin, polyvinylpyrrolidone, polyvinylpyrrolidone/vinyl acetate copolymer, crospovidone, povidone, polymethacrylates, starch, pregelatinized starch, tragacanth, dextrin, sucrose, or glucose, and the like.

Examples of disintegrants are starches, pregelatinized corn starch, pregelatinized starch, celluloses, cross-linked carboxymethylcellulose, crospovidone, cross-linked polyvinylpyrrolidone, calcium salts, a sodium alginate complex, clays, alginates, gums, or sodium starch glycolate, and any disintegrant used in tablet preparations.

Examples of diluents are lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Examples of surfactants are sodium lauryl sulfate, sorbitan monooleate, polyoxyethylene sorbitan monooleate, polysorbates, polaxomers, bile salts, glyceryl monostearate, PluronicTM line (BASF), and the like.

Examples of pH modifying agents (buffers) are citric acid, succinic acid, fumaric acid, malic acid, tartaric acid, maleic acid, glutaric acid sodium bicarbonate and sodium carbonate and the like.

Examples of stabilizers are any antioxidation agents, buffers, or acids, and the like.

Examples of suitable lubricants are magnesium stearate, calcium hydroxide, talc, sodium stearyl fumarate, hydrogenated vegetable oil, stearic acid, glyceryl behapate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

Examples of wetting agents are oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

Examples of anti-adherents (e.g., glidants) include talc, corn starch, DL-leucine, sodium lauryl sulfate, and magnesium, calcium, or sodium stearates, and the like.

Examples of pharmaceutically compatible carriers include a pharmaceutically acceptable oil, (e.g., sesame oil), water, alcohols, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

Additionally, drug formulations are discussed in, for example, Remington's, The Science and Practice of Pharmacy (2000); Lieberman, H. A. and Lachman, L., Eds., Pharmaceutical Dosage Forms, Marcel Decker, New York, N.Y., 1980; and Liebeman et al., Pharmaceutical Dosage Forms (Volumes 1-3, 1990).

When a desired excipient serves as a diluent, it can be a solid, semi-solid or liquid material, which acts as a vehicle, carrier or medium for the active ingredient. Thus, compositions suitable for use in methods of the instant invention can be in the form of a tablet, pill, powder, lozenge, sachet, cachet, troche, suspension, emulsion, aerosol (as a solid or in a liquid medium), capsule (e.g. soft and hard gelatin or HPMC capsules), sterile packaged powder, dispensable powder, granule, or solution wherein the cannabinoid molecules are substantially dissolved in the carrier component.

Tablet dosage forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents and pharmaceutically compatible carriers. In one embodiment of the present invention, the manufacturing processes may employ one or a combination of methods: (1) dry mixing, (2) direct compression, (3) milling, (4) dry or non-aqueous granulation, (5) melt granulation, or (6) fusion. Lachman et al., The Theory and Practice of Industrial Pharmacy (1986). Such tablets may also comprise film coatings, which disintegrate upon oral ingestion or upon contact with diluent.

Compressed tablets are solid dosage forms prepared by compacting a formulation containing an acid-labile pharmaceutical agent and/or buffering agent and/or excipient selected to aid the processing and improve the properties of the product. The term “compressed tablet” generally refers to a plain, uncoated tablet for oral ingestion, prepared by a single compression or by pre-compaction tapping followed by a final compression.

The tablets or pills suitable for use in methods of the present invention may be coated or otherwise compounded to provide a dosage form affording the advantage of improved handling or storage characteristics. For example, the tablet or pill can comprise an inner dosage and an outer dosage component, the latter being in the form of an envelope over the former.

Since a tablet may be used to form rapidly disintegrating tablets, chewable tablets, lozenges, troches or swallowable tablets; the intermediate formulations, as well as the process for preparing them, provide additional aspects of the present invention.

Effervescent tablets and powders may also be used in accordance with the present invention. Effervescent salts have been used to disperse medicines in water for oral administration. Effervescent salts are granules or coarse powders containing a medicinal agent in a dry mixture, usually composed of sodium bicarbonate, citric acid and tartaric acid. When the salts are added to water, the acids and the base react to liberate carbon dioxide gas, thereby causing “effervescence.”

In certain embodiments, oral dosage forms used in the present invention are room temperature stable dosage forms. In certain embodiments, these dosage forms are prepared according the methods described in United States Patent Publication No. 2006/0160888 to Kottayil, et al; the disclosure of which is hereby incorporated by reference in its entirety.

Liquid dosage forms may also be used in methods according to the present invention and include non-aqueous solutions; suitably flavored non-aqueous syrups; oil suspensions; and flavored emulsions with edible oils, such as cottonseed oil, sesame oil, coconut oil or peanut oil, as well as elixirs and similar pharmaceutical vehicles.

Many other types of release delivery systems are available and known to those of ordinary skill in the art. They include polymer-based systems, such as polylactic and polyglycolic acid, polyanhydrides and polycaprolactone; nonpolymer systems that are lipids, including sterols, such as cholesterol, cholesterol esters and fatty acids, or neutral fats, such as mono-, di- and triglycerides; hydrogel release systems; silastic systems; peptide-based systems; wax coatings; compressed tablets using conventional binders (See, for example, Lieberman et al., Pharmaceutical Dosage Forms, 2 Ed., Vol. 1, pp. 209-214 (1990), and excipients; partially fused implants; and the like. Specific examples include, but are not limited to: (a) erosional systems in which the polysaccharide is contained in a form within a matrix, found in U.S. Pat. No. 4,452,775; U.S. Pat. No. 4,667,014; and U.S. Pat. No. 4,748,034 and U.S. Pat. No. 5,239,660; and (b) diffusional systems in which an active component permeates at a controlled rate through a polymer, found in U.S. Pat. No. 3,832,253 and U.S. Pat. No. 3,854,480.

Formulations suitable for parenteral administration include aqueous and non-aqueous solutions, isotonic sterile injection solutions, which can contain anti-oxidants, buffers such as acetate and phosphate, toxicity adjusting agents, such as sodium chloride, pH adjusting agents, such as hydrochloric and phosphoric acid, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

In a preferred embodiment, dronabinol is administered according to methods of the invention as an aeorosolized formulation. Non-limiting examples of aerosolized formulations of dronabinol are disclosed in U.S. Pat. No. 6,509,005 to Peart et al., which is hereby incorporated by reference herein in its entirety.

In one embodiment, the invention is directed to methods of slowing the progression of MS in patients by administering a cannabinoid in unit dose form. In a preferred embodiment, the cannabinoid is dissolved in a liquid carrier and encapsulated in a gelatin or non-gelatin capsule. The liquid carrier may be hydrophobic, hydrophilic, or amphiphilic. The miscibility of the cannabinoid with the chosen liquid carrier may be increased with solubilizing agents, wetting agents and or surfactants.

In one embodiment of the invention, the composition comprises 2.5 mg D9-THC, gelatin glycerin, sesame oil and titanium dioxide.

In another embodiment of the invention, the composition comprises iron oxide red, iron oxide black, gelatin, glycerin, sesame oil and titanium dioxide.

In still another embodiment of the invention, the composition comprises 10 mg of D9-THC, iron oxide red, iron oxide yellow, gelatin, glycerin, sesame oil and titanium dioxide.

Dronabinol may be administered in combination with therapeutically effective amounts of one or more additional medications, for example medications used to treat symptoms of MS or to treat MS itself (disease-modifying agents). Non-limiting examples of medications that may be administered in combination with dronabinol include amantadine, baclofen, mineral oil, papaverine, meclizine (Antivert®), hydroxyzine (Atarax®), interferon-.quadrature.-1a (Avonex®), sulfamethoxazole (Bactrim®, Septra®), ciprofloxacin, (Cipro®), docusate (Colace®), glatiramer acetate (Copaxone®), pemoline (Cylert®), dantrolene (Dantrium®), desmopressin (DDAVP®), dexamethasone (Decadron®), prednisone (Deltasone®), tolterodine (Detrol®), phenytoin (Dilantin®), oxybutynin (Ditropan®), bisacodyl (Dulcolax®), venlafaxine (Effexor®), amitriptyline (Elavil®), docusate (Enemeez®), sodium phosphate, methenamine (Mandelamine®), Balcofeno, clonazepam (Klonopin®), isoniazid (Laniazid®), vardenafil (Levitra®), nitrofurantoin (Macrodantin®), psyllium hydrophilic mucilloid (Metamucil®), alprostadil, gabapentin (Neurontin®), mitoxantrone (Novantrone®), oxybutynin (Oxytrol®), nortiptyline (Pamelor®), paroxetine (Paxil®), propantheline bromide (Pro-Banthine®), alprostadil (Prostin® VR), modafinil (Provigil®), fluoxetine (Prozac®), phenazopyridine (Pyridium®), interferon-b-1a (Rebif®), glycerin (Sani-Supp®), methylprednisolone (Solu-Medrol®), carbamazepine (Tegretol®), imipramine (Tofranil®), diazepam (Valium®), sildenafil (Viagra®), bupropion (Wellbutrin®), tizanidine (Zanaflex®), and sertraline (Zoloft®).

The following examples illustrate embodiments of the present invention but should not be construed as limiting the scope of the instant invention in any way.

### DETAILED DESCRIPTION OF THE PREFERRED EMBODIMENTS

Features of the invention will become apparent in the course of the following descriptions of exemplary embodiments which are given for illustration of the invention and are not intended to be limiting thereof.

In the following examples, and throughout this specification, all parts and percentages are by weight, and all temperatures are in degrees Celsius, unless expressly stated to be otherwise. Where the solids content of a dispersion or solution is reported, it expresses the weight of solids based on the total weight of the dispersion or solution, respectively. Where a molecular weight is specified, it is the molecular weight range ascribed to the product by the commercial supplier, which is identified. Generally this is believed to be weight average molecular weight.

### EXAMPLE 1

A study was performed to assess the use of cannabis extract and delta-9-THC in treating various symptoms associated with MS in a randomized, placebo-controlled study. Patients aged 18-64 years with clinically definite or laboratory-supported multiple sclerosis who had exhibited stable disease for the previous 6 months, with problematic spasticity (Ashworth score of greater than or equal to 2 in two or more lower limb muscle groups) were included in the trial. Patients with ischaemic heart disease, those with active sources of infection, and those taking medication such as beta interferon (that could impact spasticity) were excluded.

Patients were randomly assigned to receive one of two active treatments or placebo. Active treatment consisted of either synthetic delta-9-THC (Marinol, Solvay Pharmaceuticals, Atlanta, USA) or a cannabis extract, containing delta-9-THC and cannabidiol as the main cannabinoids (Cannador, Institute for Clinical Research, IKF, Berlin, Germany). Capsules were manufactured to contain 2.5 mg of delta-9-THC equivalent, 1.25 mg of cannabidiol, and less than 5% other cannabinoids per capsule. Medication was taken twice daily, after food. All other medication was taken as usual, except other oil-based capsules which were requested to be taken separately from trial medication to avoid possible interference with absorption.

The study started with a 5-week dose titration phase. During that period, patients increased their dose by one capsule (2.5 mg delta-9-THC equivalent) twice daily at weekly intervals. If side-effects developed, patients were advised not to increase the dose, and if side-effects were considered intolerable, the dose was reduced. Weeks 6-13 constituted a plateau phase, during which participants remained on a stable dose of medication (visits 5, 6, and 7). During week 14, patients reduced their medication by one capsule twice daily each day until they were off study medication. Patients remained off trial medication during week 15, and a final assessment was undertaken at the end of this week (visit 8).

The primary outcome measure of the study was change in spasticity related to multiple sclerosis, using the Ashworth score of spasticity (See, e.g., Ashworth, B. Preliminary trial of carisoprodol in multiple sclerosis. Practitioner 1964; 192: 540-42). Assessment of the Ashworth score was made at six visits: two pre-treatment (visits 1 and 2), three during treatment (visits 5, 6, and 7), and one after discontinuation of treatment (visit 8). The Ashworth score is an assessment of biological impairment and is dependent on the estimation of the physician. The score consists of a 5-point scale (0=normal, 1=slight catch when the limb is moved, 2=anything more than a catch but not restricting movement, 3=considerable increase in tone limiting passive flexion, 4=limb rigidity in flexion or extension). Ten muscle groups on each side of the body (elbow flexors, extensors, pronators and supinators; wrist and finger flexors; hip adductors, knee flexors and extensors, and foot plantar flexors) were assessed. Each patient was assessed supine on a couch, or as close to this position as was tolerated, after resting for 15 min. The limb being assessed was moved rapidly in the direction required by assessment. As spasticity can change with passive limb movement, the number of movements of each joint was kept to a minimum. The presence of more than seven beats of clonus on examining a joint was taken as implying at least grade 2 spasticity.

Table 1 provided immediately below provides baseline characteristics of the participants.

TABLE 1

| | Cannabis | | | | | |
|---|---|---|---|---|---|---|
| | Extract | | Delta-9-THC | | Placebo | |
| | No. | Mean | No. | Mean | No. | Mean |
| Sex | | | | | | |
| Male | 76 | — | 63 | — | 78 | — |
| Female | 135 | — | 143 | — | 135 | — |
| Age (years) | 211 | 50.5 | 206 | 502 | 213 | 50.9 |
| Height (cm) | 209 | 167.5 | 205 | 167.9 | 210 | 168.0 |
| Weight (kg) (n = 630) | 211 | 71.7 | 206 | 71.2 | 213 | 71.6 |
| Body-mass Index (kg/m²) | 209 | 25.6 | 205 | 25.2 | 210 | 25.4 |
| Mean Baseline Ashworth | | | | | | |
| Upper-body muscles | 211 | 5.0 | 206 | 5.9 | 212 | 5.4 |
| Lower-body muscles | 211 | 16.8 | 206 | 16.7 | 213 | 16.1 |
| All muscle groups | 211 | 21.8 | 206 | 22.5 | 213 | 21.4 |
| Form of MS | | | | | | |
| Relapsing/remitting | 6 | 3% | 14 | 7% | 13 | 6% |
| Primary progressive | 53 | 25% | 43 | 21% | 49 | 23% |
| Secondary progressive | 152 | 72% | 149 | 72% | 151 | 71% |
| Ambulatory status | | | | | | |
| Able to walk | 103 | 49% | 95 | 46% | 105 | 49% |
| Unable to walk | 108 | 51% | 111 | 54% | 108 | 51% |

Of the 630 patients included in the intention-to-treat analysis, follow-up data on the primary outcome was obtained for 611 (97%). Completion and return of data for the secondary outcome measures was also generally high, with data available for analysis from 84-91% of patients.

With respect to analysis of Ashworth scores, 81% (n=513) of patients had the same assessor throughout or had a different assessor at just one visit (cannabis extract 82% (n=173), delta-9-THC 82% (n=168), placebo 81% (n=172)). The primary outcome was defined as the change from baseline (mean of two baseline pre-treatment visits) to the end of the 13-week treatment period (visit 7). In accordance with the protocol, missing Ashworth scores at visit 7 were replaced by carrying forward the most recent Ashworth score available during the treatment phase. In total, 39 scores were carried forward; 28 from visit 6 and 11 from visit 5, distributed across treatments (12 cannabis extract, 17 delta-9-THC, 10 placebo). Primary outcome data were not available for 46 patients originally randomized (12 cannabis extract, 19 delta-9-THC, 15 placebo).

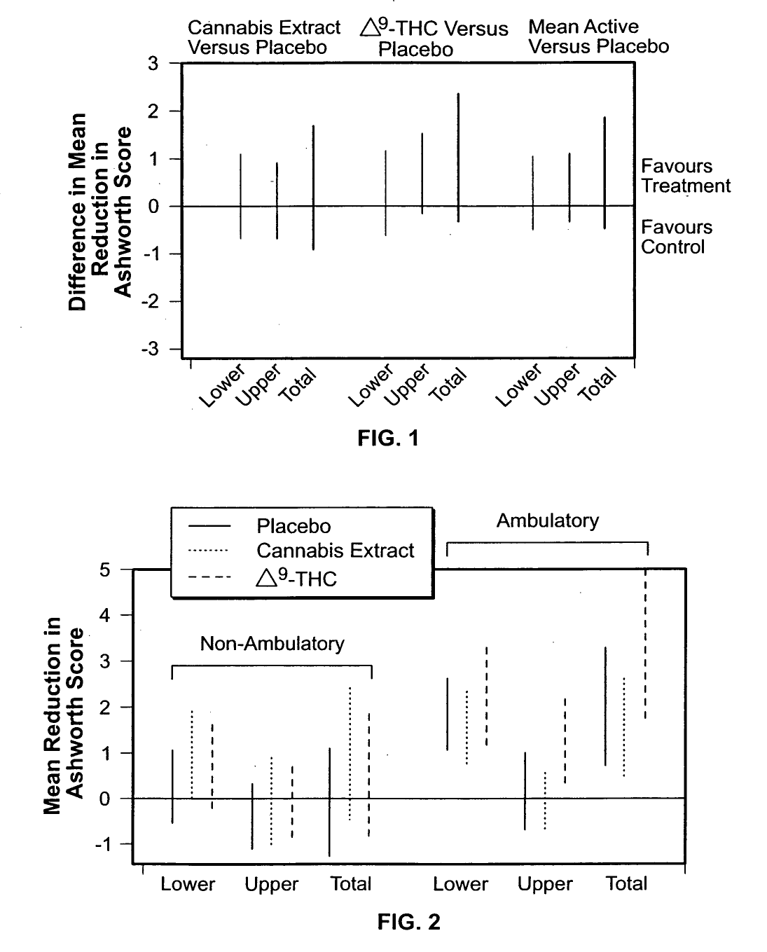

There was no statistically significant evidence of an effect of treatment on change in total Ashworth score from baseline to 13 weeks follow-up (p=0.29 with adjustment for ambulatory status and center, p=0.40 without adjustment). Mean (SD) changes in total Ashworth scores (baseline minus follow-up) were 1.24 (6.60), 1.86 (7.95), and 0.92 (6.56) for cannabis extract, delta-9-THC, and placebo, respectively. Corresponding figures for upper-body muscle groups were −0.05 (4.11), 0.48 (4.70), and −0.11 (4.04), and for lower-body muscle groups were 1.29 (4.37), 1.39 (5.21), and 1.04 (4.20). With both active treatments, an improvement over placebo was observed for the treatment effect when adjusted for center and for ambulatory status (See FIG. 1).

There was no statistically significant evidence of a treatment effect on changes in lower-body (adjusted for center and ambulatory status p=071, unadjusted p=074) or upper-body (p=020 and p=031) components of the Ashworth score, and no evidence of any interaction effect between center and treatment, between ambulatory status and treatment, or between baseline Ashworth score and treatment.

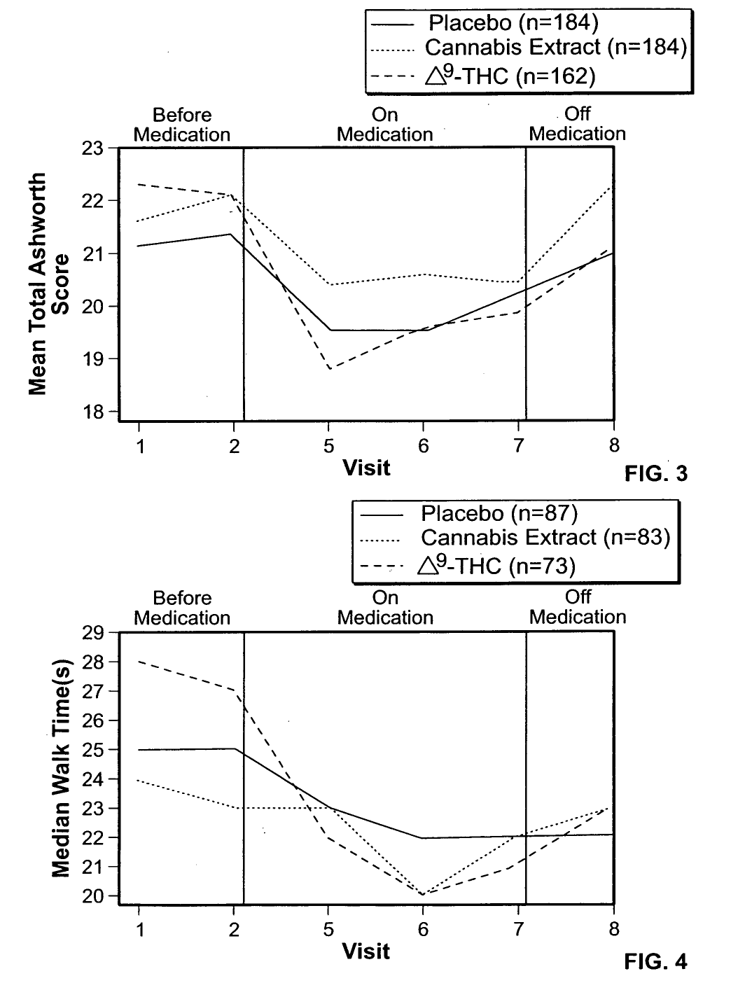

Both center (p<0.0001) and ambulatory status (p=0.002) had a significant effect on change in Ashworth score (FIG. 2). Estimated mean reduction in total Ashworth score for ambulatory patients relative to nonambulatory patients was 1.78 adjusted for treatment and center. There was also an improvement in the mean scores with treatment, occurring in all treatment groups, including placebo (FIG. 3).

### EXAMPLE 2

Secondary outcomes were also measured in the above-described study. Such secondary outcomes included the Rivermead mobility index (See e.g. Collen, F. M. et al., The Rivermead mobility index: a further development of the Rivermead motor assessment. Int. Disanil. Stud. 1991; 13:50 54) hereby incorporated by reference in its entirety, a timed 10 meter walk, four self completion questionnaires—the United Kingdom neurological disability score (See e.g. Sharrack, B., Hughes R. A., The Guy's neurological disability scale (GNDS): a new disability measure for multiple sclerosis. Mult. Sder. 1999; 5: 223-33), and a series of nine category-rating scales. For the category-rating scale assessment, patients were asked to assess how their symptoms had been over the previous week compared with how they were just before the study started. Categories included irritability, depression, tiredness, muscle stiffness, tremor, pain, sleep, muscle spasms, and amount of energy. Data are discussed below.

With respect to secondary outcome measures, 322 patients provided at least one baseline walk time. Of these, seven (1 cannabis extract, 3 delta-9-THC, 3 placebo) dropped out of the trial. Walk times were obtained from 278 patients at visit seven. In total, 20 patients were unable to walk (8 cannabis extract, 5 delta-9-THC, 7 placebo) and very large walk times were substituted for these individuals.

Overall, a significant treatment effect on walk time from baseline to visit 7 (p=0.015) was observed. The median time taken to walk 10 meters was reduced from baseline to follow-up by 12% with delta-9-THC compared with a reduction with cannabis extract of 4% and placebo of 4%. FIG. 4 shows median walk time by visit and treatment group for patients who provided walk-time information at all six assessor visits.

Category rating scales were used to assess whether patients felt their symptoms had improved while on treatment relative to before start of treatment. Data are shown in Tables 2 and 3. Overall, patients felt that symptoms of pain, sleep quality, spasms, and had improved while on active treatment. No effect was noted with respect to y, depression, tiredness, tremor, or energy.

TABLE 2
Patient Reporting of Secondary Outcomes.

| | Improvement | | | Same | | | Deterioration | | |
|---|---|---|---|---|---|---|---|---|---|
| | Cannabis | Δ-9- | | Cannabis | Δ-9- | | Cannabis | Δ-9- | |
| | Extract | THC | Placebo | extract | THC | Placebo | extract | THC | Placebo |
| Irritability | 46 | 37 | 31 | 42 | 42 | 63 | 30 | 32 | 23 |
| | (39%) | (33%) | (26%) | (36%) | (38%) | (54%) | (25%) | (29%) | (20%) |
| Depression | 43 | 36 | 38 | 44 | 47 | 64 | 33 | 42 | 35 |
| | (36%) | (29%) | (28%) | (37%) | (38%) | (47%) | (28%) | (34%) | (26%) |
| Tiredness | 46 | 35 | 36 | 51 | 46 | 79 | 68 | 76 | 52 |
| | (28%) | (22%) | (22%) | (31%) | (29%) | (47%) | (41%) | (48%) | (31%) |
| Spasticity | 95 | 89 | 67 | 43 | 40 | 52 | 46 | 47 | 64 |
| | (52%) | (51%) | (37%) | (23%) | (23%) | (28%) | (25%) | (27%) | (35%) |
| Shake/tremor | 49 | 52 | 45 | 48 | 44 | 53 | 31 | 32 | 37 |
| | (38%) | (41%) | (33%) | (38%) | (34%) | (39%) | (24%) | (25%) | (27%) |
| Pain | 68 | 64 | 42 | 48 | 43 | 58 | 32 | 22 | 42 |
| | (46%) | (50%) | (30%) | (32%) | (33%) | (41%) | (22%) | (17%) | (30%) |
| Sleep | 82 | 71 | 59 | 62 | 57 | 79 | 20 | 24 | 25 |
| | (50%) | (47%) | (36%) | (38%) | (38%) | (48%) | (12%) | (16%) | (15%) |
| Spasms | 96 | 81 | 67 | 50 | 49 | 68 | 34 | 27 | 38 |
| | (53%) | (49%) | (39%) | (28%) | (29%) | (39%) | (19%) | (22%) | (22%) |
| Energy | 61 | 61 | 45 | 73 | 63 | 78 | 49 | 49 | 61 |
| | (33%) | (35%) | (24%) | (40%) | (36%) | (42%) | (27%) | (28%) | (33%) |

### EXAMPLE 3

At visit 8 in the study described in Examples 1 and 2, patients were asked uestions about whether treatment had improved pain, tremor, spasticity, or ymptoms. Table 3 shows patient responses to those questions. Overall, more patients perceived an improvement in spasticity and pain when taking the active treatments than when taking placebo. Difference in perception of improvement in tremor was not statistically significant and no treatment effect on bladder symptoms was identified. Although there was no stratification for these specific symptoms between groups, the groups were broadly balanced for these symptoms apart from bladder symptoms, where there were fewer patients with urinary symptoms in the group taking, delta-9-THC.

There was a significant association between the actual treatment and the treating doctor's assessment of whether the patient was on active treatment (p<0.001). According to the treating doctors' assessment, 71% (n=140) of the cannabis extract group, 66% (n=119) of the delta-9-THC group, and 43% (n=85) of the placebo group were on active treatment. Similarly there was an association between the actual treatment and the patients' view of what they had taken (p<0.001). According to patients' reports, 77% (n=151), 77% (n=139), and 50% (n=98) of the cannabis extract, delta-9-THC, and placebo groups, respectively, thought that they had been on active treatment.

There was no association between the assessors' opinion of treatment and the actual treatment (p=0.72). The proportions viewed by the assessor as being on active medication in the three groups were 44% (n=90) cannabis extract, 39% (n=73) delta-9-THC, and 42% (n=86) placebo.

TABLE 3
Patient assessment of treatment benefit after week 8.

| Symptom | Cannabis Extract | Δ-9-THC | Placebo |
|---|---|---|---|
| Improvement | (n = 197) | (n = 181) | (n = 198) |
| Bladder | | | |
| Yes | 68 (44%) | 67 (40%) | 51 (33%) |
| No | 87 (56%) | 97 (59%) | 102 (67%) |
| Pain | | | |
| Yes | 83 (57%) | 64 (50%) | 51 (37%) |
| No | 63 (43%) | 64 (50%) | 86 (63%) |
| Tremor | | | |
| Yes | 58 (48%) | 44 (40%) | 43 (33%) |
| No | 64 (52%) | 67 (60%) | 89 (67%) |
| Spasticity | | | |
| Yes | 121 (61%) | 108 (60%) | 91 (46%) |
| No | 76 (39%) | 73 (40%) | 107 (54%) |

TABLE 4
Adverse events reported by patients.

| Adverse Event | Cannabis Extract | Δ-9-THC | Placebo |
|---|---|---|---|
| MS relapse or possible relapse | 1 | 1 | 7 |
| Urinary tract infection | 1 | 3 | 4 |
| Pneumonia | 1 | 2 | 1 |
| Blocked/insertion of suprapubic | 1 | 0 | 3 |
| catheter | | | |
| Constipation | 1 | 0 | 3 |
| Grand mal seizure | 1 | 0 | 1 |
| Other | 6 | 11 | 2 |

### EXAMPLE 4

In Example 4, the study described in Examples 1-3 above was continued to determine if long-term therapy with D9-TCH or cannabis plant extract would affect the progression of MS. At the end of week 15, patients were offered the opportunity to resume medication at the previously determined dose for a maximum of 52 weeks. If patients decided not to continue medication, the reasons for non-continuance were documented. Throughout the study, capsule compliance was checked at clinic visits by counting the number of capsules returned. Urine samples for cannabinoid analysis were collected at each clinic visit during the follow up phase in order to evaluate potential cannabis use in the placebo group.

In order to reduce the potential for unmasking bias from treatment side effects, two study personnel were used at each centre. A treating physician monitored the dosage, side effects, and patient wellbeing, while an assessor (usually a physiotherapist) measured the Ashworth score, timed 10 metre walk, and Rivermead Mobility Index (RMI). During this extended portion of the study, these data were collected at each of the three monthly visits. Assessors remained blinded to any discussion of dosage or side effects, and when evaluating patients' spasticity, were instructed not to have access to their assessment scores from previous visits. Self completion booklets containing the General Health Questionnaire-30 (GHQ-30), category rating scales (asking patients to assess any change in their symptoms at that point in the study compared with how they were before they started medication, and measuring irritability, depression, tiredness, muscle stiffness, tremor, pain, sleep, muscle spasms, and energy levels), the UK Neurological Disability Scale (UKNDS), and the Barthel Index (BI) were sent to participants just prior to week 27 and week 52. The Expanded Disability Status Scale (EDSS) was used at week 52.

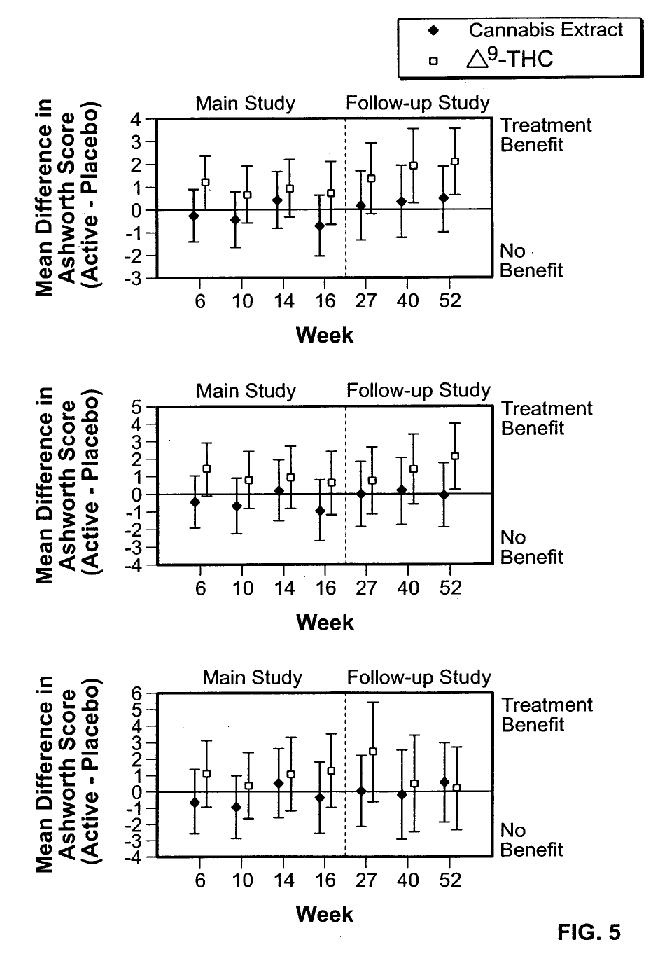

As set forth in FIG. 5, 95% Confidence intervals for differences in means (active minus placebo) of changes in total Ashworth score at each visit are shown. Positive values favour treatment. In total, 55 patients (19 on cannabis extract, 14 on Δ9-THC, and 22 placebo) started the trial towards the end of the recruitment phase for the initial 15 week study and were therefore necessarily unable to complete 52 weeks (11 visits) of follow up. For these patients, their last Ashworth score (visit 10) was carried forward as their final measured response. In FIG. 5, the top panel shows results for all patients; the middle panel shows results for patients who elected to continue medication; and the bottom panel shows results of patients who elected to discontinue medication.

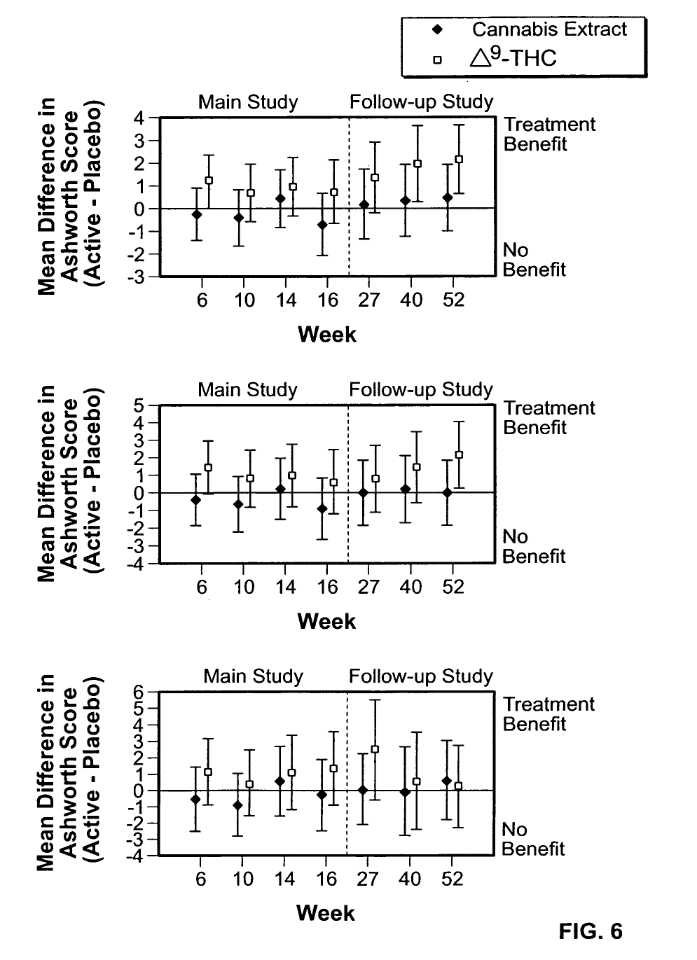

As set forth in FIG. 6, treatment effect on change in RMI adjusted for baseline, ambulatory status, and treatment centre is shown. The top panel shows results for all patients followed up; the middle panel shows results of patients followed up who chose to continue treatment; and the bottom panel shows results for patients followed up who chose not to continue treatment. Positive values favour treatment.

The primary outcome was mean change in Ashworth score from baseline to end of follow up period and was compared using an analysis of variance model, with treatments as fixed effects. Centres and ambulatory status were also added to the model.

Times taken to complete a 10 metre walk were analysed using the Kruskal-Wallis test, and non-parametric methods were used to produce confidence intervals for the median. The RMI, UKNDS, postal BI, and GHQ-30 were each analysed using non-parametric analysis of variance to compare the groups. EDSS scores were analysed using Fisher's exact test. Category rating scales were analysed using contingency table analysis. Comparison was made between patients choosing to continue on medication and those who chose to discontinue, by baseline Ashworth scores, RMI, EDSS, age, weight, ambulatory status, type of disease, sex, and use of urinary catheter. No adjustments were made for multiple comparisons.

Ashworth score data were obtained for 502 (80%) of the 630 patients in the intention to treat sample of the initial 15 week study: 154 patients on Δ9-THC (117 continued medication), 172 on cannabis extract (127 continued), 176 on placebo (111 continued). Mean (SD) difference in Ashworth scores from baseline to end of study were 1.82 (8.12), 0.10 (7.25), and 20.23 (7.87) in the Δ9-THC, cannabis extract, and placebo groups respectively. There was no evidence from baseline values that patients for whom data were missing at final assessment or those who chose not to continue medication differed from those in whom monitoring and medication continued throughout.

Comparison of the three groups using analysis of variance on the change in total Ashworth score showed evidence of a small treatment effect (p=0.01 adjusted for ambulatory status and centre, p=0.04 without adjustment) (FIG. 5).

Both centre (p,0.0001) and ambulatory status (p=0.006) had an effect on change in Ashworth score, as might be expected due to different sizes of centres and the nature of the ambulatory status, but there was no evidence of an interaction between either treatment and centre, or treatment and ambulatory status. This illustrates that the treatment effect is similar in all centres and in the two ambulatory status groups.

Suggestion of treatment effect was also found in the RMI (FIG. 6), particularly in the Δ9-THC group. This effect was strongest at the 9 month visit. No treatment effects were evident in the BI or UKNDS (all assessed using non-parametric analysis of variance to compare groups). Evidence would suggest that the RMI is the most responsive of the outcome measures used. In patients who chose to discontinue medication, there were no significant treatment effects in any of the outcome measures.

Longer term trends were also seen in the walking time data, with a reduction in walking time, followed by an increase, although statistical significance was not reached. The proportions of patients unable to walk at the end of the study, but who could walk at baseline were 6%, 14%, and 13% in the Δ9-THC, cannabis extract, and placebo groups respectively.

Rating scales showed highly significant effects on pain, spasms, spasticity, and sleep. This data is reported in Table 5 below.

TABLE 5
Table 5 Category rating scales calculated using contingency table analysis

| | Improvement | | | Same | | | Deterioration | | | |
|---|---|---|---|---|---|---|---|---|---|---|
| | Δ9-THC | CE | Placebo | Δ9-THC | CE | Placebo | Δ9-THC | CE | Placebo | p* |
| Pain (n = 356) | 30 (28) | 38 (31) | 17 (14) | 61 (56) | 53 (44) | 70 (56) | 18 (16) | 31 (25) | 38 (30) | 0.002 |
| Shaking | 25 (26) | 38 (33) | 23 (20) | 53 (55) | 56 (49) | 64 (55) | 19 (19) | 20 (18) | 30 (25) | 0.015 |
| (n = 328) | | | | | | | | | | |
| Spasms | 39 (29) | 55 (36) | 35 (23) | 62 (46) | 72 (47) | 69 (45) | 33 (25) | 25 (17) | 48 (32) | 0.002 |
| (n = 438) | | | | | | | | | | |
| Spasticity | 47 (33) | 45 (29) | 27 (17) | 48 (34) | 66 (42) | 71 (46) | 47 (33) | 45 (29) | 58 (37) | 0.004 |
| (n = 384) | | | | | | | | | | |
| Sleep | 40 (34) | 54 (38) | 36 (26) | 63 (54) | 68 (48) | 80 (58) | 14 (12) | 19 (14) | 23 (16) | 0.016 |
| (n = 397) | | | | | | | | | | |
| Energy | 41 (24) | 36 (23) | 36 (13) | 79 (56) | 87 (56) | 90 (58) | 28 (20) | 33 (21) | 46 (29) | 0.004 |
| (n = 476) | | | | | | | | | | |
| Tiredness | 32 (25) | 26 (18) | 17 (11) | 70 (54) | 81 (56) | 88 (60) | 28 (21) | 37 (26) | 43 (29) | 0.025 |
| (n = 422) | | | | | | | | | | |
| Depression | 27 (26) | 38 (34) | 20 (18) | 59 (57) | 55 (50) | 71 (65) | 17 (17) | 18 (16) | 19 (17) | 0.135 |
| (n = 324) | | | | | | | | | | |
| Irritability | 31 (30) | 32 (30) | 20 (20) | 56 (55) | 64 (60) | 61 (60) | 15 (15) | 11 (10) | 21 (20) | 0.125 |
| (n = 311) | | | | | | | | | | |

*Obtained by comparing three treatment groups on the original 11 point scale. Data are n (%) except for p values.
CE, cannabis extract

Evidence of treatment effects on ratings of shakiness, energy level, and tireness was found at 27 and 52 weeks, but not on depression or irritability, suggesting wider syptomatic benefit with time, but interpretation is difficult due to unmasking identified in the initial 15 week study period. There were no major differences between the active treatment groups in these subjective measures, in contrast to the Ashworth and RMI results.

During this study, all hospital admissions were classed as serious adverse events. Most of the 74 serious adverse events reported (24 Δ9-THC, 27 cannabis extract, 23 placebo; were as expected in this patient population. This data is reported in Table 6 below.

TABLE 6
Table 6 Frequency of serious adverse events

| | | | Cannabis | | | |
|---|---|---|---|---|---|---|
| | Δ9-THC | | extract | | | |
| | (n = 24) | | (n = 27) | | Placebo (n = 23) | |
| Adverse event | CM | DM | CM | DM | CM | DM |
| Relapse/possible | 8 | 2 | 8 | 1 | 2 | 2 |
| relapse | | | | | | |
| Urinary tract | 3 | 0 | 3 | 0 | 3tt | 1 |
| infection | | | | | | |
| Pneumonia/chest | 1 | 0 | 5 | 1 | 1 | 1 |
| infection | | | (1 died) | (died) | | (died) |
| Seizure | 0 | 0 | 1 | 0 | 1 | 2 |
| | | | (died) | | | |
| Insertion of | 0 | 0 | 1 | 1 | 0 | 0 |
| baclofen pump | | | | | | |
| Limb fracture | 0 | 0 | 1 | 0 | 1 | 0 |
| Other | 8 | 2 | 3 | 2 | 8 | 1 |
| No. of events (no. | 20 (18) | 4 (4) | 22 (21) | 5 (5) | 16 (15) | 7 (7) |
| of patients) | | | | | | |

Unblinding of treatment was not required for any event. The first phase of the study showed a reduction in relapse rate in the active treatment groups compared with placebo, as measured by the number of hospital admissions classified as serious adverse events. This was not sustained in the follow-up period. When relapses were identified from all sources (corticosteroid administration, adverse events and serious adverse events), there appeared to be a similar number of relapses in each of the three groups. This also suggests that the reduction in disability seen in the Δ9-THC group cannot be accounted for by a reduction in relapse rate.

Urinalysis for cannabinoids throughout the study demonstrated low levels of illicit cannabis use in the placebo group, with no more than four patients demonstrating urinary cannabinoid presence at any time.

### DISCUSSION

Although patients felt that both cannabis extract and Δ9-THC helped their spasticity (according to rating scales), this was confirmed objectively by the Ashworth score only in the Δ9- THC group. An improvement of approximately 2 points on the Ashworth scale from a baseline mean of around 22 occurred in this group, compared with a placebo deterioration of around 0.2. Although this change is considered to be small, clinical significance of this change from the patient perspective requires further investigation. The reasons for the differences between the two active treatment groups comparing subjective and objective measurements cannot be explained by differences in side effects, which were similar for both active groups.

In order to limit any self selection bias, data were analysed by treatment group irrespective of whether patients continued treatment. The absence of difference in characteristics between the group continuing medication and those originally randomised to receive treatment also suggests that patients continuing medication did not have a substantially different disease course. The numbers deciding to stop trial medication were similar in each of the three study arms, but the reasons given were different. Of the patients in the placebo group who decided not to continue treatment, 74% felt that the medication produced no benefit, compared with 45% in the cannabis extract and 42% in the Δ9-THC groups. Adverse effects were more commonly cited in the active treatment arms.

With respect spasticity and disability, profound effects in slowing the progression of MS were seen at the end of the 52 week treatment period in those patients receiving D9-THC. These positive results after one year were not expected by the inventor because generally the expectation of such positive results would typically require three years of treatment in order to see effects on disability in MS trials.

1. A method of slowing the progression of multiple sclerosis in a patient in need thereof comprising administration of a pharmaceutical composition comprising an effective amount of a therapeutically effective cannabinoid on a regular basis; the administration occurring over a period of time selected from the group consisting of at least about 16 weeks, at least about 27 weeks, at least about 40 weeks and at least about 52 weeks.

2. The method of claim 1, wherein the cannabinoid comprises a natural cannabinoid.

3. The method of claim 1, wherein the cannabinoid comprises a synthetic cannabinoid.

4. The method of claim 1, wherein the cannabinoid comprises a semi-synthetic cannabinoid.

5. The method of claim 1, wherein the cannabinoid is selected from the group consisting of one or more (i) natural cannabinoids that have been purified or modified; (ii) synthetically derived cannabinoids; (iii) semi-synthetic cannabinoids; (iv) esterified cannabinoids; (v) active metabolites of any of the foregoing, (vi) pro-drugs of any of the foregoing; and (vii) mixtures thereof.

6. The method of claim 1, wherein the cannabinoid comprises D9-THC.

7. The method of claim 1, wherein the cannabinoid comprises THC-hemisuccinate.

8. The method of claim 1 wherein the patient displays an improvement in at least one indicator of progression of multiple sclerosis selected from the group consisting of Ashworth score, Rivermead Mobility Index value and timed 10 metre walk compared to a group of patients administered placebo over at least about 16 weeks.

9. The method of claim 8 wherein the patient displays an improvement in Ashworth score, compared to a group of patients administered placebo over at least about 16 weeks.

10. The method of claim 8 wherein the patient displays an improvement in Rivermead Mobility index, compared to a group of patients administered placebo over at least about 16 weeks.

11. The method of claim 8 wherein the patient displays significant improvement in Rivermead Mobility index, compared to a group of patients administered placebo over at least about 16 weeks.

12. The method of claim 8 wherein the patient displays an improvement in 10 metre walk time, compared to a group of patients administered placebo over at least about 16 weeks.

13. The method of claim 8 wherein the patient displays a significant improvement in at least one symptom selected from the group consisting of pain, spasms, spasticity and sleep, compared to a group of patients administered placebo over at least about 16 weeks.

14. The method of claim 1 wherein said composition is orally administered.

15. The method of claim 14 wherein the composition is encapsulated in a gelatin or non-gelatin shell.

16. The method of claim 14 wherein the cannabinoid is present in either a liquid solution or liquid suspension.

17. The method of claim 16 wherein the liquid is a hydrophobic material, a hydrophilic material or an amphiphilic material.

18. The method of claim 17 wherein the liquid is selected from the group consisting of an oil, water or an alcohol.

19. The method of claim 18 wherein the liquid is sesame oil.

20. A method of slowing the progression of multiple sclerosis in a patient in need thereof comprising administering on a regular basis, an oral pharmaceutical composition comprising an effective amount of D9-THC, an oil and a capsule.

21. The method of claim 1 wherein the patient exhibits an increase of about 0.2 to about 1.1 in Rivermead Mobility Index values at about 12-27 weeks of administration compared to a group of patients administered placebo over at least about 16 weeks.

22. The method of claim 1 wherein the patient exhibits an increase of about 0.25 to about 1.3 in Rivermead Mobility Index values at about 25-40 weeks of administration compared to a group of patients administered placebo over at least about 16 weeks.

23. The method of claim 1 wherein the patient exhibits an increase of about 0 to about 1.2 in Rivermead Mobility Index values at about 35-50 weeks of administration compared to a group of patients administered placebo over at least about 16 weeks.

24. The method of claim 1 wherein the patient exhibits a change in Ashworth scores of from about −1 to about 2.5 at about 12-27 weeks of administration compared to a group of patients administered placebo over at least about 16 weeks.

25. The method of claim 1 wherein the patient exhibits a change in Ashworth scores of from about −5 to about 3.5 at about 25-40 weeks of administration compared to a group of patients administered placebo over at least about 16 weeks.

26. The method of claim 1 wherein the patient exhibits an increase in Ashworth scores of from about 0.2 to about 4 at about 35-50 weeks of administration compared to a group of patients administered placebo over at least about 16 weeks.

27. The method of claim 1 wherein composition is administered in a dosage form selected from the group consisting of an intranasal solution, an intranasal suspension, an inhalant solution, an inhalant suspension, a parenteral solution, a parental suspension, a transdermal patch, a transdermal gel, a transdermal cream, a transdermal ointment, and a transdermal lotion.

28. The method of claim 1 wherein the composition is administered in a dosage form selected from the group consisting of a tablet, a capsule, an oral inhalant, a nasal inhalant, an injectable, a transdermal, a sublingual, and a suppository.

29. The method of claim 1 wherein the capsule is a soft gelatin or HPMC capsule.

30. The method of claim 1 wherein the composition is administered in combination with one or more multiple sclerosis therapies.

31. The method of claim 6 wherein the composition is administered in an amount sufficient to provide from about 2.5 mg D9-THC to about 20 mg D9-THC per day.

32. The method of claim 6 wherein the composition is administered in an amount sufficient to provide about 2.5 mg D9-THC per day.

33. The method of claim 1 wherein the composition is administered about 1 to about 4 times per day.
